(19) United States
(12) Patent Application Publication
(10) Pub. No.: US 2021/0272093 A1
(43) Pub. Date: Sep. 2, 2021

(54) PRESCRIPTION PREPAYMENT WEB PLATFORM

(71) Applicant: Rite Aid, Camp Hill, PA (US)

(72) Inventors: David A. Sassaman, Middletown, PA (US); Jace M. Cresswell, New Cumberland, PA (US)

(21) Appl. No.: 17/071,076

(22) Filed: Oct. 15, 2020

Related U.S. Application Data
(60) Provisional application No. 62/983,825, filed on Mar. 2, 2020.

(51) Int. Cl. G06Q 20/28; G16H 20/10; G06Q 20/40; G06Q 20/32; G06K 19/06

(52) U.S. Cl. CPC G06Q 20/28; G16H 20/10; G06K 19/06009; G06Q 20/322; G06Q 20/4014

(57) ABSTRACT

Customer prepayment for prescription services are provided without requiring customer login through a customer account by sending to a customer an electronic prescription ready notification including a link, receiving an indication that the customer has selected the link, opening a user interface to a prepayment process accessible via the selected link, verifying an identity of the customer, presenting an indication of customer prescriptions available for pickup at a particular store location, receiving the customer's selection of a prescription from the available customer prescriptions, capturing customer regulatory compliance information including the customer's e-signature for the selected prescription, capturing customer payment information for payment authorization of a prescription order including the prescription, and upon payment authorization, providing a barcode or QR code including customer payment authorization information to the customer. A point of sale system is notified of the customer prepayment upon scanning the barcode or QR code at pickup of the prescription order.

### PRIORITY

This application claims the benefit of priority to U.S. Provisional Patent Application Ser. No. 62/983,825, filed Mar. 2, 2020, which is incorporated by reference here in its entirety.

### TECHNICAL FIELD

This application is related to systems and methods for providing prescription services and, more particularly, to a web platform that provides prescription notifications and enables customers to prepay for prescription services.

### BACKGROUND

Express pickup systems for prescriptions have been implemented in recent years to enhance customer convenience. Such systems typically require the customer to open an account with the pharmacy, download a software application provided by the pharmacy, order the prescription via the software application, prepay for the prescription online, and then use a received barcode (e.g., QR code) that may be scanned upon arrival at the pharmacy to pick up the prepaid prescription(s). However, adoption of such systems is difficult because the customers are required to open an account with the pharmacy and to provide login information. Also, such systems do not pre-collect regulatory data such as signatures, provide the customer with visibility into the customer's available prescriptions and their status, or provide the pharmacy with a notice that the customer has prepaid to expedite the prescription pickup process. Additional enhancements to express prescription pickup systems are desired to make them more user friendly and more beneficial to the pharmacies.

### SUMMARY

Various details for the embodiments of the inventive subject matter are provided in the accompanying drawings and in the detailed description text below.

The following description outlines a technique that provides customer prepayment for prescription services without requiring customer login through a customer account. The method includes sending to a customer an electronic prescription ready notification including a link, receiving an indication that the customer has selected the link and opening a user interface to a prepayment process accessible via the selected link, verifying an identity of the customer, presenting an indication of customer prescriptions available for pickup at a particular store location, receiving the customer's selection of a prescription from the available customer prescriptions, capturing customer regulatory compliance information including the customer's e-signature for the selected prescription, capturing customer payment information for payment authorization of a prescription order including the prescription, and upon payment authorization, providing a barcode (e.g., QR code) including customer payment authorization information to the customer. A point of sale system is notified of the customer prepayment upon scanning the barcode at pickup of the prescription order.

A prescription prepayment system is also described that includes a pharmacy management system at a particular store location that manages the prescription fulfillment process and sends an electronic prescription ready notification to a customer, the notification including a link, a point of sale system at the particular store location, and a server that manages prescription services to a customer, including managing customer prepayment for the prescription services without requiring the customer to login through a customer account. The server includes a memory that stores instructions and a processor that executes the instructions to perform operations including: receiving an indication that the customer has selected the link; opening a user interface to a prepayment initiation process that is accessed by the customer via the selected link; verifying an identity of the customer; presenting an indication of customer prescriptions available for pickup at a particular store location; receiving the customer's selection of one or more prescriptions from the customer prescriptions available for pickup at the particular store location; capturing customer regulatory compliance information including the customer's e-signature for the selected one or more prescriptions; capturing customer payment information for payment authorization of a prescription order including the one or more prescriptions; upon receipt of payment authorization, providing at least one of a QR code or a barcode including customer payment authorization information to the customer; and providing an order identification for the prescription order and the customer payment authorization information to the point of sale system. In sample embodiments, the point of sale system is notified of the customer payment authorization information upon pickup of the prescription order having the order identification.

The system may further include a database that stores the order identification for the prescription order and the customer payment authorization information and maintains the order identification for the prescription order and the customer payment authorization information on a store by store basis. In sample embodiments, the database periodically synchronizes the prescription order and customer payment authorization information for the particular store location with the point of sale system of the particular store location.

In sample embodiments, the processor executes the instructions to perform further operations including indicating at least one of an insurance copayment amount or a full cash price for the customer prescriptions available for pickup at the particular store location.

In other sample embodiments, the processor executes the instructions to perform further operations including assigning a GUID to the customer upon verification of the identity of the customer.

In further sample embodiments, the processor executes the instructions to perform further operations including receiving the customer's selection of prescriptions to remove from the customer prescriptions available for pickup at the particular store location.

In still further sample embodiments, the processor executes the instructions to perform further operations including receiving an indication from the customer of a person who will pick up the prescription order from the particular store location and the relationship of the person to the customer.

In yet other sample embodiments, the processor executes the instructions to perform further operations including receiving documentation of pharmaceutical care responses from the customer and tying the documentation of pharmaceutical care responses to the order identification for the prescription order.

In sample embodiments, the point of sale system may flag for presentation on a display of the point of sale system that the customer has prepaid for the prescription order having the order identification. The point of sale system may also present a PAY button to the display when a scan of the at least one QR code or barcode indicates that the customer has prepaid for the prescription order having the order identification. The point of sale system may further provide access to a pre-paid order management portal application that displays active prescription orders for the particular store location.

In other sample embodiments, a messaging server sends the electronic prescription ready notification to the customer as an SMS text message or an email including the link. A uniform resource locator (URL) shortener may also be provided to shorten a URL for the link prior to inserting the link into the SMS text message.

As discussed herein, the logic, commands, or instructions that implement aspects of the methods described herein may be provided in a computing system including any number of form factors for the computing system such as desktop or notebook personal computers, mobile devices such as tablets, netbooks, and smartphones, client terminals and server-hosted machine instances, and the like. Another embodiment discussed herein includes the incorporation of the techniques discussed herein into other forms, including into other forms of programmed logic, hardware configurations, or specialized components or modules, including an apparatus with respective means to perform the functions of such techniques. The respective algorithms used to implement the functions of such techniques may include a sequence of some or all of the electronic operations described herein, or other aspects depicted in the accompanying drawings and detailed description below. Such systems and computer-readable media including instructions for implementing the methods described herein also constitute sample embodiments.

This summary section is provided to introduce aspects of the inventive subject matter in a simplified form, with further explanation of the inventive subject matter following in the text of the detailed description. This summary section is not intended to identify essential or required features of the claimed subject matter, and the particular combination and order of elements listed this summary section is not intended to provide limitation to the elements of the claimed subject matter. Rather, it will be understood that the following section provides summarized examples of some of the embodiments described in the Detailed Description below.

### BRIEF DESCRIPTION OF THE DRAWINGS

In the drawings, which are not necessarily drawn to scale, like numerals may describe similar components in different views. The drawings illustrate generally, by way of example, but not by way of limitation, various embodiments discussed in the present document.

FIG. 1 illustrates an overview of a prescription prepayment web platform in a sample embodiment.

FIG. 2 illustrates the initiation of the prepayment process by the pharmacy and the order engine.

FIG. 3 illustrates the prepayment process in a sample embodiment.

FIG. 4 illustrates the process performed at the POS system when the customer or the customer's designee arrives to pick-up the prepaid prescription.

FIG. 5 illustrates a prescription notification in the form of a text including a link to open a user interface for interacting with a server for initiating the prepayment process.

FIG. 6 illustrates a prescription notification in the form of an email including a link to open a user interface for interacting with a server for initiating the prepayment process.

FIG. 7 illustrates a user interface that asks the customer for verification information.

FIG. 8 illustrates a display presented to the customer to identify the prescriptions that are in will call status and ready for pick up.

FIG. 9 illustrates a display prompting the customer with a signature block to electronically sign for the prescription(s).

FIG. 10 illustrates an order summary presented to the customer for prepayment.

FIG. 11 illustrates a display presented to the customer for entry of payment information.

FIG. 12 illustrates a payment confirmation notice that includes the order number for presentation to the customer once the prepayment process has been completed.

FIG. 13 illustrates an interface of the POS system including a PAY button indicating that the customer has used the prepayment option for their order and that the tender is available.

FIG. 14 illustrates an interface of the POS system showing a split tender message when additional items besides the prepaid prescription(s) are being purchased by the customer (or other designee) picking up the prescription.

FIG. 15 illustrates a link to a portal application on the POS system that lists all of the customers who have prepaid for their orders.

FIG. 16 illustrates an interface of the portal application identifying the prepaid orders ready to be picked up.

FIG. 17 illustrates a simplified flow chart of a method of prepaying for prescriptions in a sample embodiment.

FIG. 18 illustrates a block diagram of an example of a machine upon which one or more embodiments may be implemented.

### DETAILED DESCRIPTION

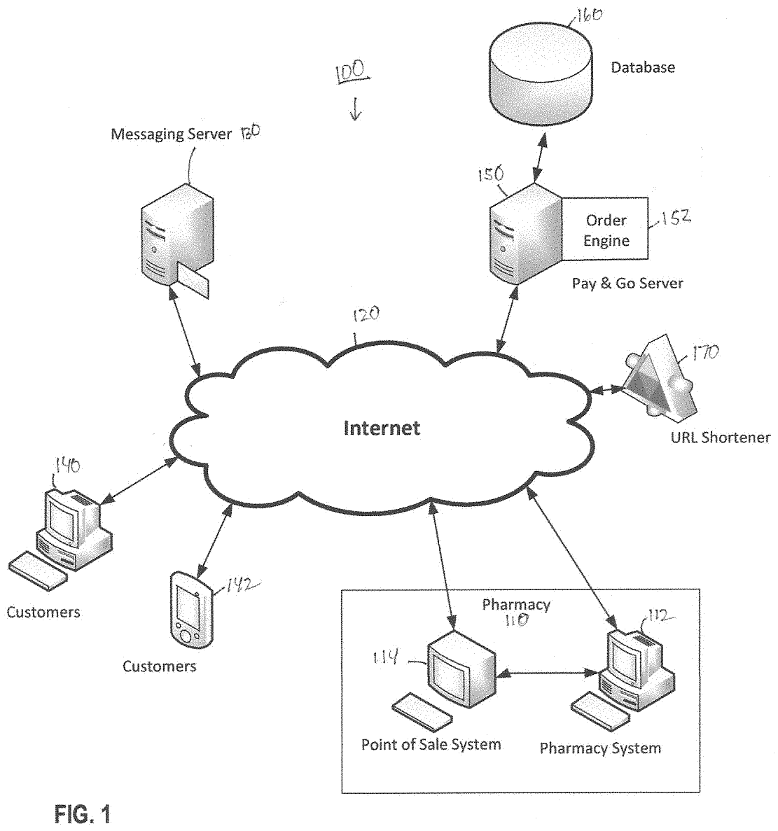

The following description with respect to FIGS. 1-18 sufficiently illustrates specific embodiments to enable those skilled in the art to practice them. Other embodiments may incorporate structural, logical, process, and other changes. Portions and features of some embodiments may be included in, or substituted for, those of other embodiments. Embodiments set forth in the claims encompass all available equivalents of those claims. The example embodiments are presented for illustrative purposes only and are not intended to be restrictive or limiting on the scope of the disclosure or the claims presented herein.

The functions described herein may be implemented in software in one embodiment. The software may consist of computer executable instructions stored on computer readable media or computer readable storage device such as one or more non-transitory memories or other type of hardware-based storage devices, either local or networked. Further, such functions correspond to modules, which may be software, hardware, firmware, or any combination thereof. Multiple functions may be performed in one or more modules as desired, and the embodiments described are merely examples. The software may be executed on a digital signal processor, ASIC, microprocessor, or other type of processor operating on a computer system, such as a personal computer, server, or other computer system, turning such computer system into a specifically programmed machine.

### Process Overview

The prescription prepayment web platform (including a web service and web user interface) described herein enables a customer to complete a prescription order online, provide the required regulatory signatures, provide customer information for the purchase, and prepay for the prescription order. The prescription order may then be delivered to the customer or the customer may pick up the order using a barcode (e.g., QR code). At the time of pick up, the point of sale register may recognize that the prescription has been prepaid when the prescription label is scanned. The resulting process provides enhanced convenience to the customer and streamlined checkout for the pharmacy.

In sample embodiments, the prescription prepayment services described herein are initiated by the pharmacist's pharmacy management system by providing a “Your Prescription is Ready” notification to the customer. The notification may be sent as an SMS text message or may be sent via other message channels or types such as an ‘Rx Waiting’ message sent via email and/or automated phone services. The customer's SMS text message or email message may include a URL link for customer selection along with text encouraging the customer to select the URL link to ‘SAVE TIME.’ In sample embodiments, a URL shortening service may be utilized to shorten the URL and to provide a web preview image to select. If enabled by the customer's mobile phone, the web preview image may be presented for customer selection. The URL link launches a user interface upon selection.

The user interface presents informational verbiage to the customer to guide the customer through the data collection process. In sample embodiments, the customer is verified before the available prescription(s) are presented. Once verified, the customer is presented with a list of prescriptions that are in will call status. In sample embodiments, only will call scripts specific to the store of the pharmacist that triggered the notification are displayed. The customer next selects which prescriptions they would like to include in the order. The customer is presented with counseling text and the phone number of the store. The customer may be asked to confirm their relationship with the person designated to pick up the prescription(s). In addition, depending on the medication, state, or agency, the customer may receive additional prompts through the user interface and/or when the customer picks up the prescription(s) at the store. The customer is also prompted to provide their electronic signature for the prescription.

The customer is provided an order summary through the user interface and given the option to ‘Cancel’ or ‘Check Out.’ Prescriptions may be removed from the order by, for example, selecting a ‘trash can’ icon. The customer pay (Check Out) amount is dynamically updated as prescriptions are removed from the order. Once the customer elects to ‘Check Out,’ the customer is taken to a credit card processor, and the customer enters their credit card information. If enabled by the customer's mobile phone, the credit card details may be pre-populated into the credit card processor. Once the customer selects ‘Pay,’ the data is submitted to a payment processor, and the customer's credit card is pre-authorized for the amount submitted.

Once the payment process has completed, the customer receives confirmation of the order inclusive of the order number. Store information is provided for reference including a phone number and a link to the hours that the store is open. Optionally, the user interface may open a new window to a store locator that uses the store number to show the store on a map. The customer then receives a confirmation text message with a barcode that the customer may use to validate a delivery or present at the store to pick up the prescription.

In the case of customer pickup, when the customer arrives at the store to pick up the prepaid prescription, store personnel may either scan the barcode or scan prescription labels. In either case, a ‘Pay’ button is presented on the point of sale register for any prepaid prescription to indicate that the tender is available. The “Pay’ button is selected to complete the sale or the store personnel may review the online prescription prepaid order in a management portal application prior to the label scan. Only active orders will display in the management portal application. When the sale is completed, the order will no longer be searchable. At this time, the prescriptions are retrieved, the management portal application is closed, and the prescription labels may be scanned in the conventional manner. The prepaid prescriptions are systematically matched as they are scanned.

The store personnel finalizes the transaction by selecting the ‘Pay’ button and the credit card is charged. If additional prescriptions or additional items are added to the transaction, a message will appear for a split tender and the customer will be required to pay the balance. The point of sale register also may prompt for additional customer interactions as necessary.

### System Overview

FIG. 1 illustrates an overview of a prescription prepayment web platform 100 for implementing the prescription prepayment services in a sample embodiment. As illustrated, the store housing the pharmacy 110 includes a pharmacy system 112 that the pharmacist uses to fill prescriptions and to initiate sending of the “Your Prescription is Ready” notification to the customer. The pharmacy 110 also includes a point of sale system 114 including one or more point of sale registers that may be used to complete the sale when the customer arrives to pick up the prepaid subscription.

In sample embodiments, the “Your Prescription is Ready” notification is provided via the Internet 120 to a messaging server 130 controlled by the store or an associated entity. The messaging server generates the customer notification as an SMS text message or may send the customer notification via other message channels or types such as an ‘Rx Waiting’ message sent via email and/or automated phone services. The customer notification is received by one or more customer devices 140, 142. In the case of an SMS text message or an email message, the customer may initiate the prepayment services by selecting a URL link in the SMS text message or email message that opens a user interface and directs the customer to a “Pay & Go” server 150 that drives the prepayment services.

In sample embodiments, the prepayment services are driven by an order engine 152 on server 150 that guides the customer through the data collection process. The transaction data is stored in database 160 (e.g., SQL database) along with other relevant store, customer, and prescription information. The order engine 152 executes software for verifying the customer and providing a list of prescriptions that are in will call status to the customer via the customer device 140 or 142. The order engine 152 also may call a URL shortening service 170 to shorten the URL to be included in the message to the customer device 140 or 142.

The customer's selection of the URL opens a user interface on the customer device 140 or 142 for interaction with the order engine 152. The user interface provides access to a software script run by the order engine 152 to verify the customer's identity and to present the available prescriptions to the customer for the customer's selected pharmacy location (prescriptions in will call status for the selected pharmacy). Upon receipt of a customer selection of one or more prescriptions, the order engine 152 further executes code to present the customer with counseling text and the phone number of the store and optionally may ask the customer to confirm her relationship with the person designated to pick up the prescription. In addition, depending on the medication, state, or agency, the customer may receive additional prompts through the user interface. The order engine 152 further executes code to prompt the customer to provide their electronic signature for the prescription and to present an order summary. Upon receipt of the customer's selection of the option to ‘Check Out,’ the order engine 152 directs the customer to a credit card processor where the customer's credit card is pre-authorized for the amount submitted.

Once the payment process has completed, the order engine 152 provides the customer with confirmation of the order inclusive of the order number. Store information is provided for reference including a phone number and a link to the hours that the store is open, and a new window to a store locator may be provided showing the store on a map. Finally, the order engine 152 may provide the customer with a confirmation text message with a barcode either directly or via the messaging server 130. In sample embodiments, the customer may use the barcode to validate a delivery or present at the store to pick up the prescription.

The collected information is retained in tables in the database 160 and provided to the POS system 114 at the selected store through a periodic near-real-time synchronization operation (e.g., every few minutes) for saving until the customer picks up the prescription or the prescription is delivered.

### Customer Devices

The prescription prepayment web platform described herein is designed to work with the customer's communication devices 140, 142 such as smart phones, laptops, personal computers, and the like without requiring the customer to download specific software or to have an account with the pharmacy. Rather, as noted below, the prescription prepayment web platform drives the process of collecting customer information, authenticating the customer, collecting payment information, and authorizing prescription pickup or delivery via the customer's communication devices without requiring the user to download particular software. All that is required is Internet access to the web interface provided by the prescription prepayment web platform. The process is then driven by the order engine 152.

### Pharmacy System

The pharmacy system 112o functions in the conventional fashion. A pharmacy system 112 such as the Nexgen Pharmacy Management Application implemented by Rite Aid Corporation provides a system for prescription order fulfillment and customer notification. The pharmacy system 112 issues notifications in the conventional way to initiate the processes described herein. However, in sample embodiments, the messaging server 130 inserts a URL link into the notifications in order to initiate the prepayment process described herein.

### Point of Sale System

The point of sale (POS) system 114 completes the sale of the prescription to the customer. In sample embodiments, applications of the point of sale system 114 interface with the pharmacy system 112. For example, the point of sale system 114 supports the GetRxInfo service. GetRxInfo is a webservice called by a POS application of the POS system 114 when a prescription is scanned at the POS system 114 during a sale. The GetRxInfo service returns the data needed by the POS application of the POS system 114 to sell the prescription. Examples of the data needed by the POS application of the POS system 114 include the customer's date of birth, whether the customer's ID needs to be collected before the script can be sold, etc. The POS application of the POS system 114 calls the GetRxInfo webservice by passing the location, prescription (Rx) number, and date of service for the script that is to be sold. The query parameters are passed to the GetRxInfo service in a query string of the URL.

The response from the GetRxInfo service will be either a success message along with the data needed to sell the prescription or an error message if the prescription is not in a state to be sold. A successful response will be sent back to the client if the prescription is in will call status and POS data is available in the pharmacy system 112. However, an error response may be sent back in following cases: the prescription is not found in the pharmacy system 112, the prescription is found in the pharmacy system 112 but is not in will call status, or the prescription is in will call status but POS data is not available in the pharmacy system 112. The error response may include any or all of the following data elements: version number for the service, prescription location, prescription number, date of service, pharmacy system order number for the prescription, pharmacy system order item number for the prescription, response status, and error message. If the message is a success message, the success message may further include the pharmacy system customer number, customer name and birth date, customer pay amount for the prescription, whether tax has to be collected from the customer for the prescription and, if so, the amount of the tax. The success message also may identify if the payment is by cash, by commercial insurance plan, or by government backed insurance plan. If by insurance plan, the success message may also identify the insurance agency, plan and group.

The success message may also contain certain POS flags including, for example, the drug being dispensed and a prompt for a customer HIPAA acknowledgement message and customer authorization request for customer enrollment in certain programs at the POS. The flags may also identify if the customer is in a certain program such as a senior program or a prescription refill program. In sample embodiments, the flags further include an indication as to whether the customer has prepaid for the order and whether the relationship of the customer to the person picking up the order has been collected. Flags may also be provided at the POS system 114 to initiate other types of data collection from the customer that need not be described in further detail here.

### Pay & Go Server 150

The Pay & Go Server 150 may be adapted to implement the prescription prepayment web platform. In a sample embodiment, the Pay & Go Server 150 implements a customer verification service and obtains a list of prescriptions for the verified customer. Also, an order engine 152 drives the customer through the data collection and prepayment process.

### Customer Verification and Get List of Prescriptions

A customer verification service verifies the customer's last name and date of birth and provides a customer valid flag and a customer ID. The customer verification service receives the following input parameters in a JavaScript Object Notation (JSON) format: a GUID of the customer, date of birth, last name, store number, and service type. For example:

```
{
  “GuID”: “46b324487d774eafa0cab5ae7d17b483”,
  “StoreNumber” : “11025”,
  “LastName” : “ROMAN”,
  “DateOfBirth” : “1974/02/01”,
  “ServiceType”: “pickup”
}
```

The output attributes will also be in a JSON format and include: customer is valid, customer, list of addresses, list of prescriptions, list of location on a map, and “Success” or “Error” status. If an error message is provided, the output may also include the error message.

### Example

```
{
  “Status”: “Success”,
  “address”: [
    {
      “ID”: “”,
      “addr1”: “200 NEWBERRY COMMONS”,
      “addr2”: “TEST TEST3”,
      “cty”: “ETTERS”,
      “st”: “PA”,
      “zipCde”: “173197485”,
      “postalCde”: “”,
      “country”: “US”
    },
    {
      “ID”: “”,
      “addr1”: “200 NEWBERRY CMNS”,
      “addr2”: “”,
      “cty”: “ETTERS”,
      “st”: “AK”,
      “zipCde”: “173190000”,
      “postalCde”: “”,
      “country”: “US”
    },
    {
      “ID”: “”,
      “addr1”: “200 NEWBERRY COMMONS”,
      “addr2”: “TEST TEST333”,
      “cty”: “ETTERS”,
      “st”: “PA”,
      “zipCde”: “173190000”,
      “postalCde”: “”,
      “country”: “US ”
  “customerValid”: true,
  “prescriptions”: [
    {
      “profId”: “0”,
      “locn”: “5078”,
      “rx”: “1344”,
      “dteOfSrvc”: “04032019”,
      “ndc”: “00093423301”,
      “ndcDesc”: “BUMETANIDE 1 MG TABLET”,
      “dysSply”: “30”,
      “qty”: “30.0”,
      “rflNbr”: “0”,
      “patPayAmt”: “0.0”,
      “dosgFrm”: “”,
      “adjInd”: “P”,
      “NPI”: “1801892187”,
      “RxProcessId”: “500”,
      “RxStatusInd”: “”,
      “Selectable”: “Y”
    },
    {
      “profId”: “1”
      “locn”: “5078”,
      “rx”: “1343”,
      “dteOfSrvc”: “04032019”,
      “ndc”: “00006057761”,
      “ndcDesc”: “JANUMET 50-1,000 MG TABLET”,
      “dysSply”: “30”,
      “qty”: “30.0”,
      “rflNbr”: “0”,
      “patPayAmt”: “0.0”,
      “dosgFrm”: “”,
      “adjInd”: “P”,
      “NPI”: “1801892187”,
      “RxProcessId” “500”,
      “RxStatusInd”: “”,
      “Selectable”: “Y”
    }
  ],
  “customer”: {
    “GUID”: “46b324487d77eafa0cab5ae7d17b483”,
    “1stNme”: “ROMAN”,
    “frstNme”: “CHRISTINE”,
    “midInit”: “”,
    “dteOfBrth”: “02011974”,
    “gender”: “F”
    “cstNbr” “225003025”
  }
}
```

### Submit Order

On the other hand, a submit order request to the Pay & Go Server 150 provides details to save/update an order service from the API of the order engine 152. The submit order request may be in JSON format and may include:
Store Number
Customer Number
GUID
Service Type
Array prescriptions (conditional)
   Location
   Prescription Number
   Date of Service
Channel (communication channel)
Document of Pharmaceutical Care
Signature Data
Relationship
Mailing Address
   Name
   Street 1
   Street 2
   City
   State
   Zip
Payment Info
   Card Type
   Approval Code
   Payment Timestamp
      The resulting output is the order ID.

### Get Barcode

In sample embodiments, a barcode may be generated that is provided to the customer for the customer to present at the time of prescription pickup. For example, a getQRCode instruction may be used to generate a QR code based on the input data. The output will be a PNG image of the QR code that is provided to the customer's smart phone in an HTML wrapper that displays the QR Code. Corresponding techniques may be used to generate a barcode instead of a QR code.

### Order Engine 152

The order engine API is a set of functions for retrieval and update of order information. In sample embodiments, the web service supports JSON and XML, as the data interchange response format and supports JSON as the request format. As will be explained below, the order engine 152 supports a number of services including saving an order, updating an order, updating an order status, and finalizing a delivery order.

### Save Order

The save order service receives an order and saves it to the database 160 (FIG. 1). The order contains the prescriptions and most of the information about the order. An order number is generated and returned to the caller. In a sample embodiment, the following request parameters may be sent in the body of the request:
Order Type (conditional)
Store Number (conditional)
Customer Number (prescription) (conditional)
IP Address (conditional)
Origin (conditional) (e.g., mobile app, customer service, web application, e-commerce application, etc.)
Customer Service Agent ID
Pick-Up Location (store number)
Array Prescription (conditional)
   Pay Amount
   Location (store number) for array prescription
   Prescription Number
   Date of Service
Documentation of Pharmaceutical Care (DPC) (conditional) (e.g., did not ask, declined counseling, counseled, etc.)
Relationship (conditional) (patient, parent, spouse, caregiver, etc.)
Signature Data (conditional)
Patient Date of Birth (required)
Address (required)
   First Name (required)
   Last Name (required)
      NOTE: If any of the conditional parameters shown below are included in the request, then all of the conditional parameters are required. Street2 is always optional.
Street1 (conditional)
Street2 (optional)
City (conditional)
State (conditional)
Zip (conditional)
Country (conditional)
Latitude (conditional)
Longitude (conditional)
Wellness Number (optional)
Phone (conditional)
Payment Information (conditional based on Order Number)
Card Type
Approval Code
Payment Timestamp
Transaction ID
Order Total

### Examples

```
JSON: (request)
{
  “OrderNbr”: “12345-6”,
  “OrderType”: “VENDOR”,
  “StoreNumber”: 5078,
  “CustomerNumber”: “123456789”,
  “IPAddress“: “255.255.255.255”,
  “Origin”: “MOB”,
  “CSAgent”: “TEST”,
  “PickUpLocation”: “5078”,
  “ArrayRx”: [
      {
        “PayAmount”: 123.45,
        “Locn”: “5078”,
        “RxNumber”: “402”,
        “DateOfService”: “2019-06-20”
      }
  ],
  “DPC”: 0,
  “Relationship”: “Patient”,
  “SignatureData”: “AAECAwQFBgcICQoLDA0ODw==”,
  “Address”: {
      “FirstName”: “John”,
      “LastName”: “Doe”,
      “Street1”: “200 Snowy Tree Ln”,
      “Street2”: “”,
      “City”: “Etters”,
      “State”: “PA”,
      “Zip”: “17319”,
      “Country”: “USA”,
      “Latitude”: “40.157614”,
      “Longitude”: “−76.819351”
  },
  “WellnessNumber”: “12345”,
  “Phone”: “7177612633”,
  “PaymentInfo”: [
      {
        “CardType”: “VISA”,
        “ApprovalCode”: “123456”,
        “PaymentTimestamp”: “2019-06-
21T12:01:34.618”,
        “TransactionId”: “TEST”,
        “OrderTotal”: 16.99
      }
  ]
}
JSON: (response)
{
  “ErrCde”: “”,
  “ErrMsg”: “”,
  “ErrTyp”: “”,
  “Status”: “Success”,
  “Data”: {
    “OrderNbr”: “12345-6”
  }
}
```

### Update Order

The update order service receives an order and saves it to the database 160 (FIG. 1). The order contains the prescriptions and most of the information about the order. Each object can be updated individually. If one prescription is added to an order, all prescriptions in the order may be supplied. The response may include any errors, a description of the error, the exception type, if any, a status indicator (e.g., “success” or “error”), and associated order number data including the order number and the number of prescriptions on the order. The update order service will search on all matching criteria to attempt to find a wellness card. In a sample embodiment, the following request parameters may be sent in the body of the request:
Order Number (required)
Order Type (conditional)
Store Number (conditional)
Customer Number (prescription) (conditional)
IP Address (conditional)
Origin (conditional) (e.g., mobile app, customer service, web application, e-commerce application, etc.)
Customer Service Agent ID (optional)
Pick-Up Location (optional) (store number)
Array Prescription (conditional)
   Pay Amount
   Location (store number) for array prescription
   Prescription Number
   Date of Service
Document of Pharmaceutical Care (conditional) (e.g., did not ask, declined counseling, counseled, etc.)
Relationship (conditional) (patient, parent, spouse, caregiver, etc.)
Signature Data (conditional)
Patient Date of Birth (required)
Address (required)
   First Name (required)
   Last Name (required)
      NOTE: If any of the conditional parameters shown below are included in the request, then all of the conditional parameters are required. Street2 is always optional.
Street1 (conditional)
Street2 (optional)
City (conditional)
State (conditional)
Zip (conditional)
Country (conditional)
Latitude (conditional)
Longitude (conditional)
Wellness Number (optional)
Phone (conditional)
Payment Information (conditional based on Order Number)
Card Type
Approval Code
Payment Timestamp
Transaction ID
Order Total

### Examples

```
JSON: (request)
{
  “OrderNbr”: “12345-6”,
  “OrderType”: “VENDOR”,
  “StoreNumber”: 5078,
  “CustomerNumber”: “123456789”,
  “IPAddress”: “255.255.255.255”,
  “Origin”: “MOB”,
  “CSAgent”: “TEST”,
  “PickUpLocation”: “5078”,
  “ArrayRx”: [
      {
        “payAmount”: 123.45,
        “Locn”: “5078”,
        “RxNumber”: “402”,
        “DateOfService”: “2019-06-20”
      }
  ],
  “DPC”: 0,
  “Relationship”: “Patient”,
  “SignatureData”: “AAECAwQFBgcICQoLDA0ODw==”,
  “Address”: {
      “FirstName”: “John”,
      “LastName”: “Doe”,
      “Street1”: “200 Snowy Tree Ln”,
      “Street2”: “”,
      “City”: “Etters”,
      “State”: “PA”,
      “Zip”: “17319”,
      “Country”: “USA”,
      “Latitude”: “40.157614”,
      “Longitude”: “−76.819351”
  },
  “WellnessNumber”: “12345”,
  “Phone”: “7177612633”,
  “PaymentInfo”: [
      {
        “CardType”: “VISA”,
        “ApprovalCode”: “123456”,
        “PaymentTimestamp”: “2019-06-
21T12:01:34.618”,
        “TransactionId”: “TEST”,
        “OrderTotal”: 16.99
      }
  ]
}
JSON: (response)
{
  “ErrCde”: “”,
  “ErrMsg”: “”,
  “ErrTyp”: “”,
  “Status”: “Success”,
  “Data”: [
    “OrderNbr”: “12345-6”
  }
}
```

### Update Order Status

The update order status service updates the current order status of the provided order number to the provided order status. The query parameters include order number and, optionally, format (e.g., JSON or XML). The request parameters sent in the request body may include the status of the order (e.g., order created, order paid, waiting for customer/driver pickup, shipped, canceled, sold/delivered, etc.). The response may include any errors, a description of the error, the exception type, if any, a status indicator (e.g., “success” or “error”), and associated order number data including the order number and the number of prescriptions on the order.

### Examples

```
JSON: (request)
{
  “OrderNbr”: “12345-6”,
  “Status”: “Delivered”
}
JSON: (response)
{
  “ErrCde”: “”,
  “ErrMsg”: “”,
  “ErrTyp”: “”,
  “Status”: “Success”,
  “Data”: {
    “OrderNbr”: “12345-6”
  }
}
```

### Finalize Delivery Order

The finalize delivery order webservice receives the data collected during sale of prescription(s) and saves the data in the pharmacy system 112. The data includes an electronic image of the customer's signature, Documentation of Pharmaceutical Care (DPC), and the relationship of the customer paying for the prescription or picking up the prescription for the customer. The response may include any errors, a description of the error, the exception type, if any, a status indicator (e.g., “success” or “error”), and associated order number data including the order number and the number of prescriptions on the order. The request parameters sent in the request body may include:
Order Number (required)
Order Type (conditional)
Store Number (required) for the pharmacy that sold the script(s)
Array Prescription (required)
   Prescription Number
   Date of Service
   Documentation of Pharmaceutical Care (i.e. indicates whether the customer was counseled, declined counseling, or was not asked)
   Relationship of the customer picking up, receiving, or paying for the script to the patient (e.g., patient, parent, spouse, caregiver, other, etc.)
Image (required)
   Electronic image of customer's signature
   Image format (e.g., PNG)
Transaction (optional)
   Register Number identifying the register where the script(s) were sold
   Sale Transaction Number ale transaction number
   Transaction date/time—The date and time of the sale transaction
Delivery (optional)
   Delivery Date/Time—The date and time the order was delivered to the customer.

### Examples

```
JSON: (request)
{
  “OrderNbr”: “12345-6”,
  “OrderType”: “VENDOR”,
  “StoreNumber”: 5078,
  “ArrayRx”: [
      {
        “RxNumber”: 12345,
        “DateOfService”: “2019-06-26”,
        “DPC”: 1,
        “Relationship”: “Patient”
      }, {
        “RxNumber”: 12346,
        “DateOfService”: “2019-06-26”,
        “DPC”: 1,
        “Relationship”: “Patient”
      }
  ],
  “Image”: {
      “data”: “AAECAwQFBgcICQoLDA0ODw==”,
      “format”: “png”
  },
  “Transaction”: {
      “reg-nbr”: 1,
      “tran-nbr”: 123,
      “tran-datetime”: “2019-04-05T01:01:01”
  },
  “
  “dlvy-datetime”: “2019-04-05T01:01:01”
}
JSON: (response)
{
  “Status”: “Success”,
  “ErrCde”: “”,
  “ErrTyp”: “”,
  “ErrMsg”: “”,
  “Data”: {
    “OrderNbr”: “12345-6”
  }
}
```

### Process Flow

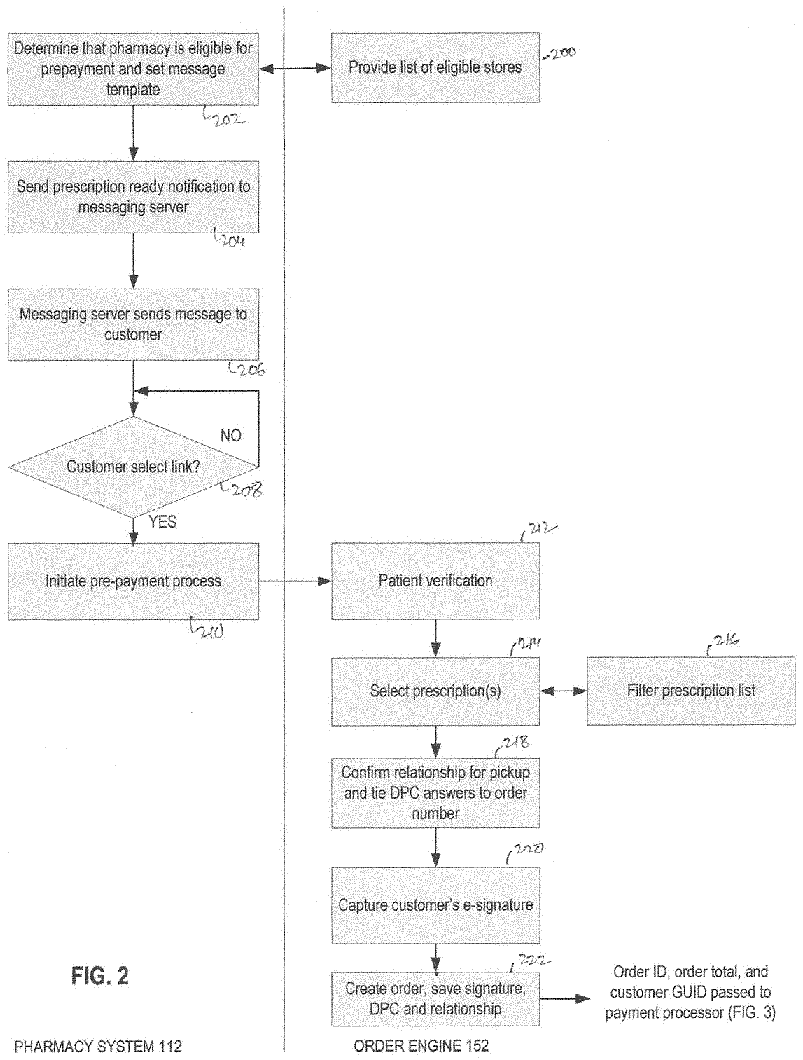

FIG. 2 illustrates the initiation of the prepayment process by the pharmacy system 112 and the order engine 152. As illustrated, the order engine 152 provides a list of available stores at operation 200 to the pharmacy 110 so that the pharmacy system 112 may determine at operation 202 whether the pharmacy is eligible for prepayment (i.e., is participating in the prepayment system) and sets the message template of the pharmacy system 112 accordingly. Once the requested prescription is ready, the pharmacy system 112 provides a “prescription ready” notification to the messaging server 130 at operation 204. The messaging server 1330 then sends a message to the customer at operation 206 indicating that the prescription is ready. As described in more detail below, the “prescription is ready” notification includes a URL link that the customer may select to initiate the prepayment process. If the customer selects the link at operation 208, the prepayment process is initiated at operation 210 by contacting the order engine 152 through a user interface that is initiated by the customer's device 140 or 142.

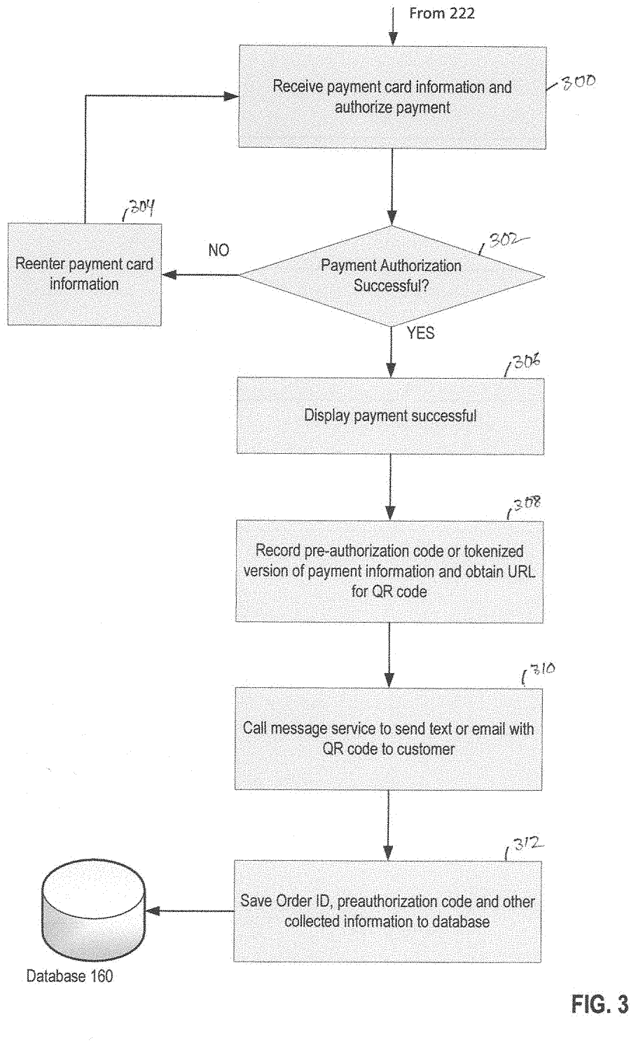

At operation 212, the order engine 152 requests information from the customer to verify the customer's identity. Once the customer is verified, the available prescriptions are presented to the customer for selection at operation 214. As appropriate, the prescription list is filtered at operation 216 to present only those prescriptions that are will call prescriptions specific to the pharmacy that triggered the customer notification. The list of prescriptions also may be updated as the customer elects to cancel or remove prescriptions. The order engine 152 may provide data to the web platform 100, which presents screens to the customer at operation 218 whereby the customer may confirm the relationship of the person who will be picking up the prescription. Also, Documentation of Pharmaceutical Care (DPC) questions are also presented to the customer at operation 218 and the customer's answers are tied to the order number. The order engine 152 then presents screens for capturing the customer's e-signature at operation 220. The order engine 152 also creates the order for the selected prescriptions and saves in database 160 the customer's signature, DPC responses, and the relationship of the person picking up the prescription. The order ID, order total, and customer GUID is passed to the payment processor at operation 222 for processing as described with respect to FIG. 3.

FIG. 3 illustrates the prepayment process in a sample embodiment. The order ID, order total, and customer GUID is received along with payment card information at operation 300. The information is passed to a payment processor for payment authorization. If the payment authorization is not successful at operation 302, the customer is asked to reenter the payment card information at operation 304 prior to repeating the payment authorization process at operation 300. If the payment authorization is successful, a notification is sent to the customer's display at 306 indicating that the payment was successful. A pre-authorization code or tokenized version of the payment information is recorded at operation 308, and a URL is obtained for a barcode including the pre-authorization code or tokenized version of the payment information. At operation 310, the message service implemented by message server 130 is called to send a text or email including the barcode to the customer. The order ID, pre-authorization code, and other information collected from the customer is stored in database 160 at operation 312. The order is now prepaid and ready for customer pickup.

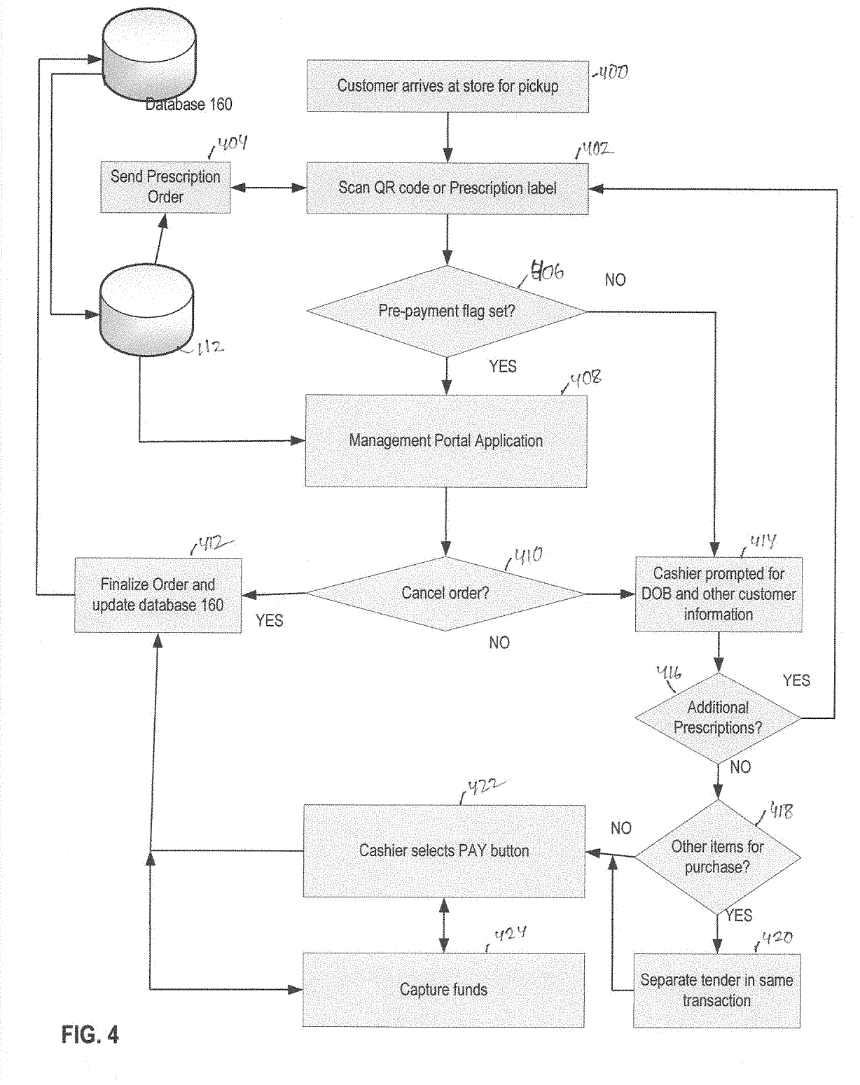

FIG. 4 illustrates the process performed at the POS system 114 when the customer or the customer's designee arrives to pick-up the prepaid prescription at 400. The store personnel scan the customer's barcode or the prescription label at operation 402. The prescription order identified from the barcode or prescription label is sent to the prescription system 112 at operation 404 to obtain the prescription information using the GetRxInfo service described above. The POS system 114 checks at operation 406 whether a prepayment flag has been set. If so, the POS system 114 may open the management portal application at operation 408 to pull from the pharmacy system 112 the prepayment information that has been pushed down to the pharmacy system 112 from the database 160 during a periodic synchronization operation. If the order is canceled at operation 410, the order is finalized and the database 160 is updated accordingly at operation 412. Otherwise, the store personnel (e.g., cashier) is prompted for the date of birth and other customer information to authenticate the customer at operation 414.

Once the customer has been authenticated at operation 414, the POS system 114 searches at operation 416 for additional prescriptions. If additional prescriptions are found, the process returns to operation 402 to scan the barcode or the prescription label of the additional prescription(s). Otherwise, the POS system 112 prompts the store personnel to check for other items for purchase at operation 418. If other items are to be purchased, a separate tender is generated in the same transaction at operation 420. The payment process is finalized at operation 422 when the cashier selects the ‘pay’ button. The customer's funds are then captured from, for example, the credit card authorization system at operation 424. The order is finalized and the database 160 is updated at operation 412. The purchase is now completed and the customer may leave with the customer's purchases.

Customer Interaction with Pay & Go Server

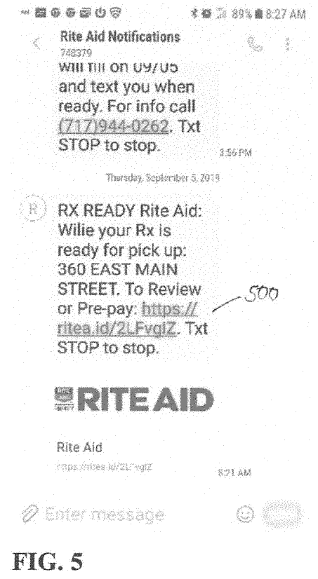

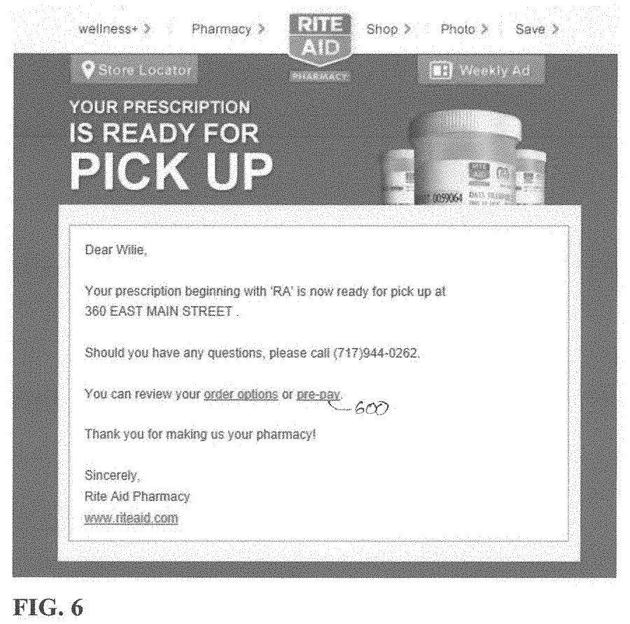

In sample embodiments, customers who are signed up for prescription notifications will have an option to prepay for their pharmacy order. The prescriptions may be submitted online or via phone for delivery customers (the prepayment may include any delivery costs and avoid a DPC receipt), drive-thru customers, and pickup customers. After the pharmacist fills a prescription and places the prescription in will call status in the pharmacy system 112, customers with prescription notifications will receive either a text as shown in FIG. 5 or an email as shown in FIG. 6 indicating that their “Prescription is Ready” and to prepay for their order to save time at the time of pickup of the prescription. When the customer selects to prepay by selecting link 500 or link 600, a user interface will launch directing the customer to complete the payment process by interacting with the Pay & Go server 150. The following steps are completed using the user interface to interact with the Pay & Go server 150:

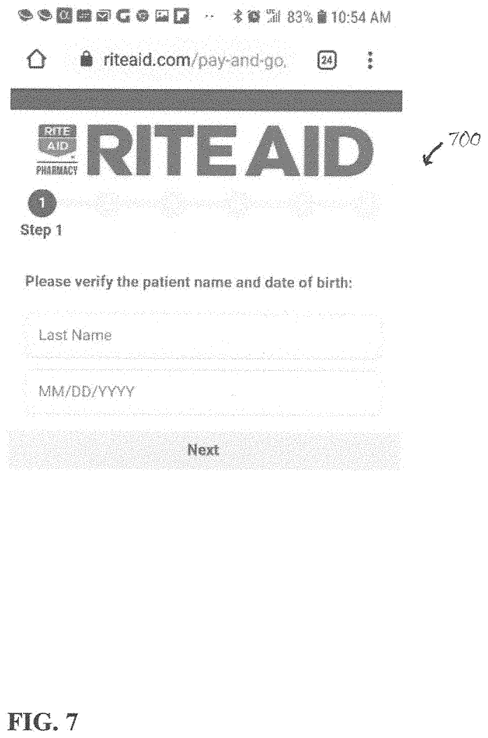

STEP 1: Informational verbiage is presented to the customer via the user interface, and the customer is verified before being presented with the available prescriptions. As shown in FIG. 7, the user interface 700 asks the customer to complete her last name (exactly as it appears on their prescription label) and date of birth. Upon completion, the customer hits “NEXT.”

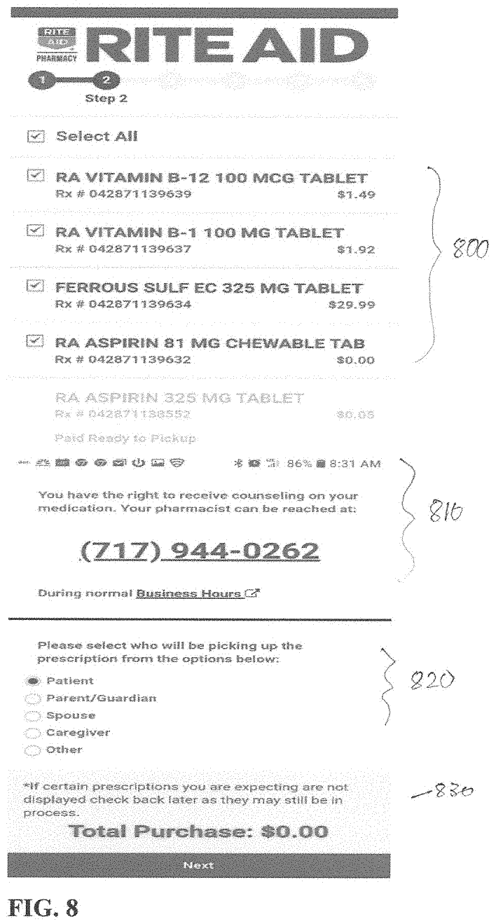

STEP 2: As illustrated in FIG. 8, the customer will see which prescriptions 800 are in will call status and ready for pick up. In sample embodiments, only the will call prescriptions specific to the pharmacy that triggered the customer notification are displayed. The customer then selects all or individual prescriptions that she would like to include in her order. The customer is presented with counseling text and store phone number at 810 so that the customer may contact her pharmacist if she has any questions. The customer also selects who will be picking up the prescriptions and confirms the relationship of the designated person at 820. Depending upon the medication, state, or agency, there also may be additional prompts that the customer would normally receive at the pharmacy when picking up the prescription. The total purchase price is listed at the bottom of the screen as shown at 830. The customer then hits “NEXT.”

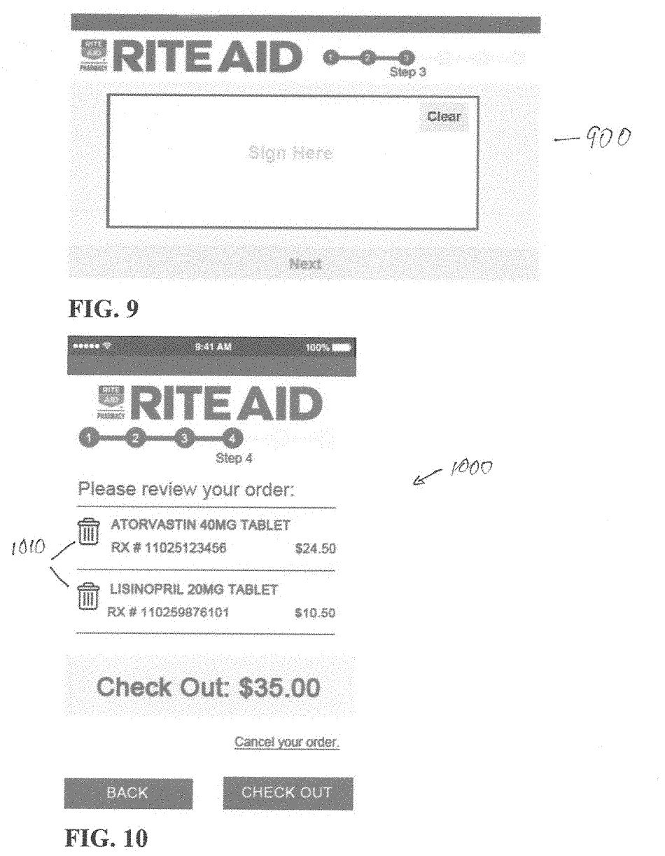

STEP 3: As shown in FIG. 9, the customer is prompted with a signature block 900 to electronically sign for the prescriptions (this replaces the DPC signature on the sig cap).

STEP 4: The customer receives the order summary 1000 as shown in FIG. 10 and is given the option to go back, cancel, or check out. Optionally, the customer may remove prescriptions by, for example, selecting a trash can icon 1010. The customer pay (checkout) amount dynamically updates as prescriptions are removed from the order.

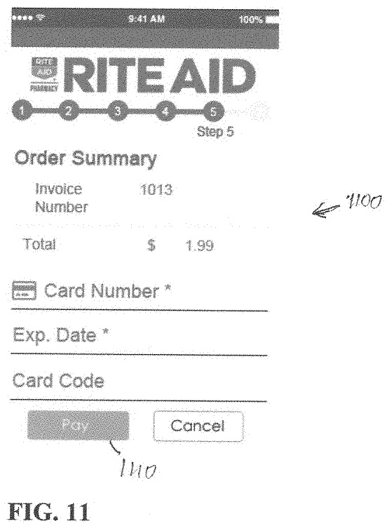

STEP 5: The customer is taken to a credit card processor and is asked to enter payment information (e.g., credit card or debit card information) into interface 1100 as shown in FIG. 11. Some of the information may pre-populate depending on the customer's smart phone. Once the information has been entered, the customer selects “PAY” button 1110 to submit the payment data for authorization. The credit card is pre-authorized for the amount submitted.

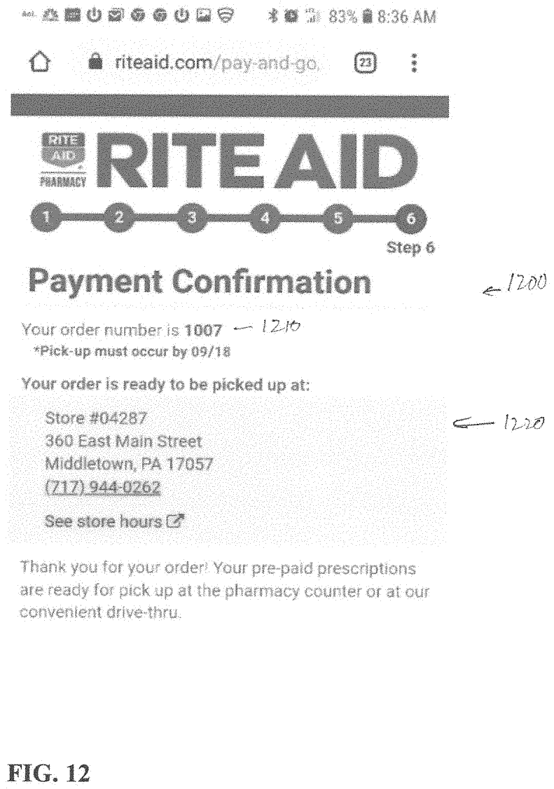

STEP 6: As shown in FIG. 12, the customer receives a payment confirmation notice 1200 that includes the order number 1210. Store information 1220 is provided for reference including the phone number and a link to the hours that will open a new window to a store locator, which passes the store number. The customer will then receive a confirmation text message or email with a barcode that may be used to facilitate pickup of the prepaid prescription.

### In-Store Pickup

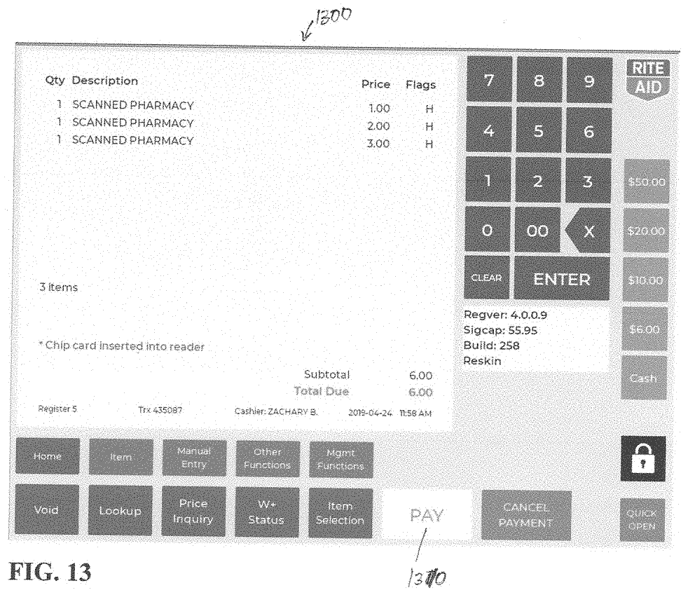

When the customer (or other designee) comes in to pick up the order, the store personnel scans the prescription label and confirms the customer's date of birth. The prepaid prescriptions are systematically matched as they are scanned. The interface 1300 of the POS system 114 then presents a “PAY” button 1310 as shown in FIG. 13 indicating that the customer has used the prepayment option for her order and that the tender is available. The store personnel finalizes the transaction by selecting the “PAY” button 1310 to charge the credit card to complete the sale. The store personnel may also review the online prescription prepaid order in a portal application prior to the label scan via an online prescription order management portal application that is provided at the POS system 114 to enable the store personnel to view orders and order details. Only active orders will display in the application, and when the sale is completed, the order will no longer be searchable. A signature capture device captures any further data that is required for the prescription if not already captured by the web platform to the pharmacy system. Optionally, the prescriptions may be retrieved, the application closed, and the prescription labels scanned in the conventional fashion.

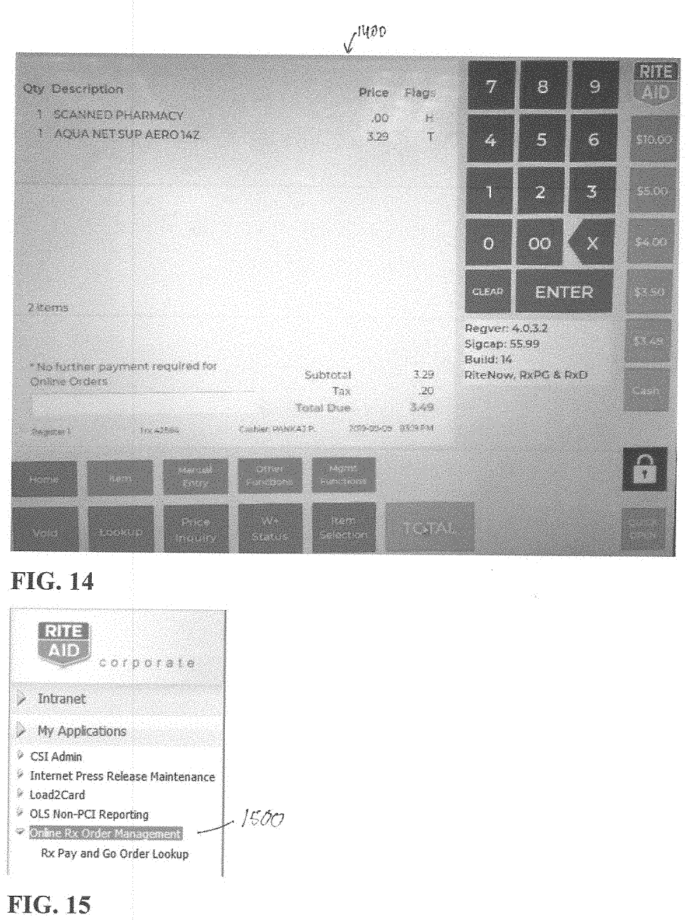

If additional items besides the prepaid prescription(s) are being purchased by the customer (or other designee) picking up the prescription, a message 1400 will appear for a split tender as shown in FIG. 14. If the customer would like to add an additional prescription or other items to the order, the additional items are scanned after scanning the prescription. The “PAY” button 1310 is hit to complete the sale for the prepay items, and the store personnel collects tender for the remaining balance. The POS system 114 may also prompt for additional customer interactions as in conventional POS systems.

If a loyalty (wellness) card is presented by the customer, the store personnel scans the prescription and then scans the loyalty card. If the customer only has her their phone number, the store personnel cancels payment and the customer is asked to enter her phone number on the sig cap. The store personnel presses total and card payment, selects online order, and presses the “PAY” button 1310 to complete the sale. The collected signature(s) may then be forwarded to the pharmacy system 112 for storage.

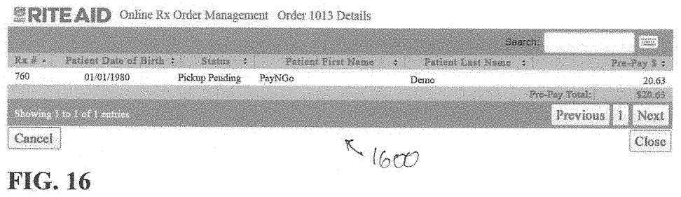

To determine if a customer has prepaid, each pharmacy has access to a portal application on the POS system 114 via a link 1500 (FIG. 15) that lists all of the customers who have prepaid for their orders on an interface 1600 like that illustrated in FIG. 16. In addition to the portal application, whether a customer has prepaid may be determined by hitting the “PAY” button 1310 that appears after scanning the prescription. To speed up the check-out process, a will call section may be provided in the pharmacy for those customers who have prepaid.

### Process Summary

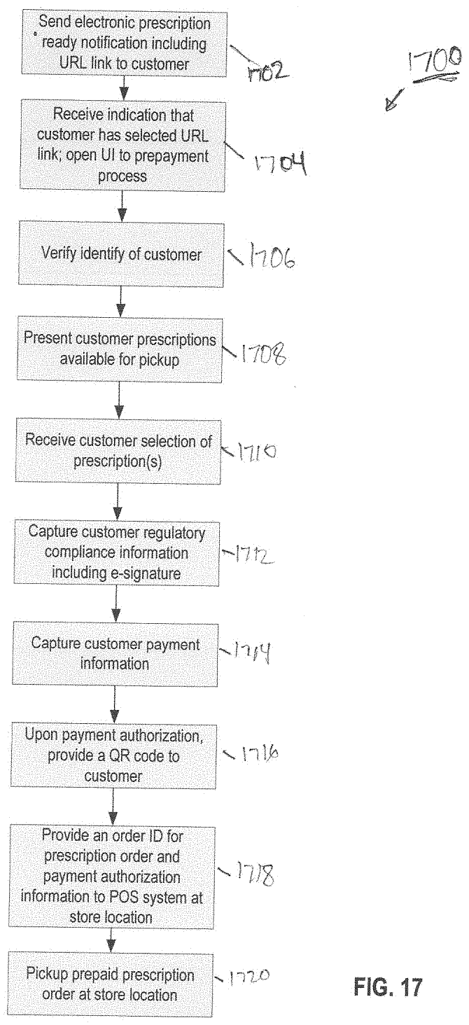

FIG. 17 illustrates a simplified flow chart of a method 1700 of prepaying for prescriptions in a sample embodiment. As illustrated, the method 1700 enables customer prepayment for prescription services without requiring the customer to login through a customer account. The method 1700 includes sending an electronic prescription ready notification to a customer at operation 1702. In sample embodiments, the notification includes a URL link for selection by the customer. At operation 1704, an indication that the customer has selected the link is received. A user interface to a prepayment initiation process is opened and the prepayment initiation process is accessed by the customer via the selected URL link. At operation 1706, the identity of the customer is verified. Once the customer has been verified, the customer is presented at operation 1708 with an indication of customer prescriptions available for pickup at a particular store location. The customer then selects one or more prescriptions from the customer prescriptions available for pickup at the particular store location, and the customer's selections are received at operation 1710.

Once the customer prescription selections have been received, customer regulatory compliance information including the customer's e-signature for the selected prescription(s) is captured at operation 1712. The customer's payment information is captured at operation 1714 and, upon payment authorization, a barcode may be provided to the customer at operation 1716. As noted above, the barcode may include customer payment authorization information for a particular prescription order. At operation 1718, an order identification for the prescription order and the customer payment authorization information is provided to a point of sale system 114 at the particular store location. Then, when the customer picks up the prepaid prescription at the particular store location, the point of sale system 114 is notified of the customer payment authorization information for the prescription order having the order identification at operation 1720.

### System Configuration

Techniques described herein may be used with one or more of the computer systems described herein and/or with one or more other systems. For example, the various procedures described herein may be implemented with hardware or software, or a combination of both. For example, the processor, memory, storage, output device(s), input device(s), and/or communication connections discussed below can each be at least a portion of one or more hardware components. Dedicated hardware logic components can be constructed to implement at least a portion of one or more of the techniques described herein. For example, and without limitation, such hardware logic components may include Field-programmable Gate Arrays (FPGAs), Program-specific Integrated Circuits (ASICs), Program-specific Standard Products (ASSPs), System-on-a-chip systems (SOCs), Complex Programmable Logic Devices (CPLDs), etc. Applications that may include the apparatus and systems of various aspects can broadly include a variety of electronic and computer systems. Techniques may be implemented using two or more specific interconnected hardware modules or devices with related control and data signals that can be communicated between and through the modules, or as portions of an application-specific integrated circuit. Additionally, the techniques described herein may be implemented by software programs executable by a computer system. As an example, implementations can include distributed processing, component/object distributed processing, and parallel processing. Moreover, virtual computer system processing can be constructed to implement one or more of the techniques or functionality, as described herein.

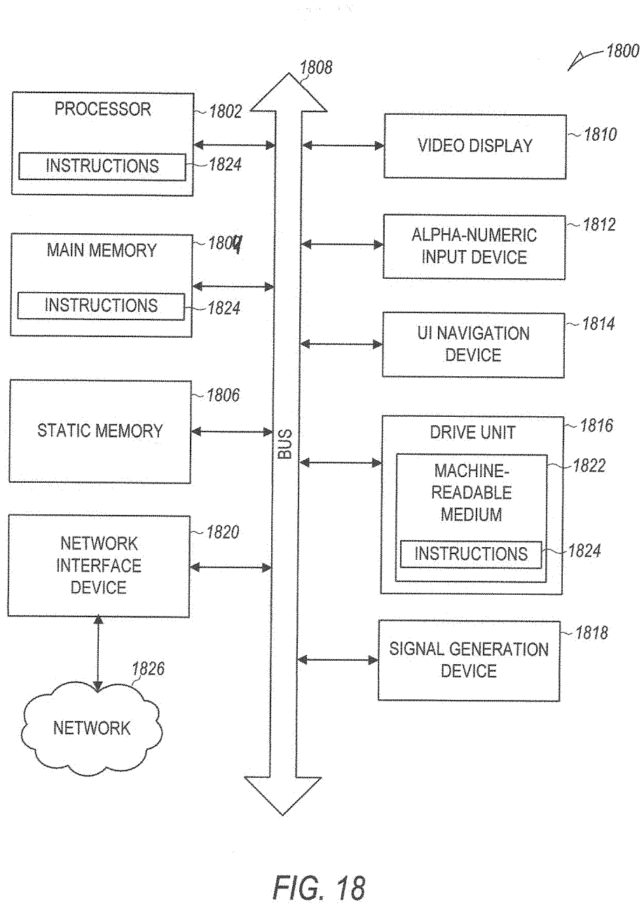

FIG. 18 illustrates a block diagram of an example of a machine 1800 upon which one or more embodiments may be implemented. In alternative embodiments, the machine 1800 may operate as a standalone device or may be connected (e.g., networked) to other machines. In a networked deployment, the machine 1800 may operate in the capacity of a server machine, a client machine, or both in server-client network environments. In an example, the machine 1800 may act as a peer machine in peer-to-peer (P2P) (or other distributed) network environment. In sample embodiments, the machine 1800 may be used in embodiments of the Pay & Go server 150 as well as the user devices 140 and 142, point of sale system 114, and pharmacy system 112 and may be a personal computer (PC), a tablet PC, a set-top box (STB), a personal digital assistant (PDA), a mobile telephone, a smart phone, a web appliance, a server, a network router, switch or bridge, or any machine capable of executing instructions (sequential or otherwise) that specify actions to be taken by that machine. For example, machine 1800 may serve as a workstation, a front-end server, or a back-end server of a communication system. Machine 1800 may implement the methods described herein (e.g., FIGS. 2-4 and 17) by running software that includes instructions that, when processed, implement the methods described herein. Further, while only a single machine 1800 is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate on, processors, logic, or a number of components, modules, or mechanisms (herein “modules”). Modules are tangible entities (e.g., hardware) capable of performing specified operations and may be configured or arranged in a certain manner. In an example, circuits may be arranged (e.g., internally or with respect to external entities such as other circuits) in a specified manner as a module. In an example, the whole or part of one or more computer systems (e.g., a standalone, client or server computer system) or one or more hardware processors may be configured by firmware or software (e.g., instructions, an application portion, or an application) as a module that operates to perform specified operations. In an example, the software may reside on a machine readable medium. The software, when executed by the underlying hardware of the module, causes the hardware to perform the specified operations.

Accordingly, the term “module” is understood to encompass a tangible hardware and/or software entity, be that an entity that is physically constructed, specifically configured (e.g., hardwired), or temporarily (e.g., transitorily) configured (e.g., programmed) to operate in a specified manner or to perform part or all of any operation described herein. Considering examples in which modules are temporarily configured, each of the modules need not be instantiated at any one moment in time. For example, where the modules comprise a general-purpose hardware processor configured using software, the general-purpose hardware processor may be configured as respective different modules at different times. Software may accordingly configure a hardware processor, for example, to constitute a particular module at one instance of time and to constitute a different module at a different instance of time.

Machine (e.g., computer system) 1800 may include a hardware processor 1802 (e.g., a central processing unit (CPU), a graphics processing unit (GPU), a hardware processor core, or any combination thereof), a main memory 1804 and a static memory 1806, some or all of which may communicate with each other via an interlink (e.g., bus) 1808. The machine 1800 may further include a display unit 1810 (shown as a video display), an alphanumeric input device 1812 (e.g., a keyboard), and a user interface (UI) navigation device 1814 (e.g., a mouse or pen). In an example, the display unit 1810, input device 1812 and UI navigation device 1814 may be a touch screen display. In sample embodiments of the machine 1800, the input device 1812 may include a microphone and/or a video recorder. The machine 1800 may additionally include a mass storage device (e.g., drive unit) 1816, a signal generation device 1818 (e.g., a speaker), a network interface device 1820, and one or more sensors 1822. Example sensors 1822 include one or more of a global positioning system (GPS) sensor, compass, accelerometer, temperature, light, camera, video camera, sensors of physical states or positions, pressure sensors, fingerprint sensors, retina scanners, or other sensors. The machine 1800 may include an output controller 1824, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The mass storage device 1816 may include a machine readable medium 1822 on which is stored one or more sets of data structures or instructions 1824 (e.g., software) embodying or utilized by any one or more of the techniques or functions described herein. The instructions 1824 may also reside, completely or at least partially, within the main memory 1804, within static memory 1806, or within the hardware processor 1802 during execution thereof by the machine 1800. In an example, one or any combination of the hardware processor 1802, the main memory 1804, the static memory 1806, or the mass storage device 1816 may constitute machine readable media.

While the machine readable medium 1826 is illustrated as a single medium, the term “machine readable medium” may include a single medium or multiple media (e.g., a centralized or distributed database, and/or associated caches and servers) configured to store the one or more instructions 1824. The term “machine readable medium” may include any medium that is capable of storing, encoding, or carrying instructions for execution by the machine 1800 and that cause the machine 1800 to perform any one or more of the techniques of the present disclosure, or that is capable of storing, encoding or carrying data structures used by or associated with such instructions. Non-limiting machine readable medium examples may include solid-state memories, and optical and magnetic media. Specific examples of machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; Random Access Memory (RAM); Solid State Drives (SSD); and CD-ROM and DVD-ROM disks. In some examples, machine readable media may include non-transitory machine-readable media. In some examples, machine readable media may include machine readable media that is not a transitory propagating signal.

The instructions 1824 may further be transmitted or received over communications network 1826 using a transmission medium via the network interface device 1820. The machine 1800 may communicate with one or more other machines utilizing any one of several transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®), IEEE 802.15.4 family of standards, a Long Term Evolution (LTE) family of standards, a Universal Mobile Telecommunications System (UMTS) family of standards, peer-to-peer (P2P) networks, among others. In an example, the network interface device 1820 may include one or more physical jacks (e.g., Ethernet, coaxial, or phone jacks) or one or more antennas to connect to the communications network 1826. In an example, the network interface device 1820 may include a plurality of antennas to wirelessly communicate using at least one of single-input multiple-output (SIMO), multiple-input multiple-output (MIMO), or multiple-input single-output (MISO) techniques. In some examples, the network interface device 1820 may wirelessly communicate using Multiple User MIMO techniques.

In the above Detailed Description, various features may be grouped together to streamline the disclosure. However, the claims may not set forth every feature disclosed herein as embodiments may feature a subset of the features. Further, embodiments may include fewer features than those disclosed in a particular example. Also, although the subject matter has been described in language specific to structural features and/or methodological acts, it is to be understood that the subject matter defined in the appended claims is not necessarily limited to the specific embodiments, features, or acts described above. Rather, the specific embodiments, features, and acts described above are disclosed as example forms of implementing the claims. Thus, the following claims are hereby incorporated into the Detailed Description, with a claim standing on its own as a separate embodiment.

What is claimed is:

1. A method of providing prescription services including customer prepayment for the prescription services without requiring customer login through a customer account, comprising:
sending an electronic prescription ready notification to a customer, the notification including a link;
receiving, by at least one processing device, an indication that the customer has selected the link, and opening a user interface to a prepayment initiation process that is accessed by the customer via the selected link;
verifying, by the at least one processing device, an identity of the customer;
presenting, by the at least one processing device, an indication of customer prescriptions available for pickup at a particular store location;
receiving, by the at least one processing device, the customer's selection of one or more prescriptions from the customer prescriptions available for pickup at the particular store location;
capturing, by the at least one processing device, customer regulatory compliance information including the customer's e-signature for the selected one or more prescriptions;
capturing, by the at least one processing device, customer payment information for payment authorization of a prescription order including the one or more prescriptions;
upon receipt of payment authorization, providing, by the at least one processing device, at least one of a QR code or a barcode including customer payment authorization information to the customer; and
providing, by the at least one processing device, an order identification for the prescription order and the customer payment authorization information to a point of sale system at the particular store location, whereby the point of sale system is notified of the customer payment authorization information upon pickup of the prescription order having the order identification.

2. A method as in claim 1, further comprising storing, by the at least one processing device, the order identification for the prescription order and the customer payment authorization information in a database that maintains the order identification for the prescription order and the customer payment authorization information on a store by store basis.

3. A method as in claim 2, further comprising the database periodically synchronizing prescription order and customer payment authorization information for the particular store location with a point of sale system of the particular store location.

4. A method as in claim 1, wherein presenting the indication of customer prescriptions available for pickup at the particular store location comprises indicating, by the at least one processing device, at least one of an insurance copayment amount or a full cash price for the customer prescriptions available for pickup at the particular store location.

5. A method as in claim 1, wherein verifying the identity of the customer includes assigning, by the at least one processing device, a GUID to the customer upon verification of the identity of the customer.

6. A method as in claim 1, wherein receiving the customer's selection of one or more prescriptions from the customer prescriptions available for pickup at the particular store location includes receiving, by the at least one processing device, the customer's selection of prescriptions to remove from the customer prescriptions available for pickup at the particular store location.

7. A method as in claim 1, further comprising receiving, by the at least one processing device, an indication from the customer of a person who will pick up the prescription order from the particular store location and the relationship of the person to the customer.

8. A method as in claim 1, further comprising receiving, by the at least one processing device, documentation of pharmaceutical care responses from the customer and tying the documentation of pharmaceutical care responses to the order identification for the prescription order.

9. A method as in claim 1, further comprising the point of sale system flagging for presentation on a display of the point of sale system that the customer has prepaid for the prescription order having the order identification.

10. A method as in claim 9, further comprising the point of sale system presenting a PAY button when a scan of the at least one QR code or barcode indicates that the customer has prepaid for the prescription order having the order identification.

11. A method as in claim 1, further comprising providing access to a pre-paid order management portal application via the point of sale system, the pre-paid order management portal application displaying active prescription orders for the particular store location.

12. A method as in claim 1, wherein sending the electronic prescription ready notification to the customer comprises sending to the customer an SMS text message or an email including the link.

13. A method as in claim 12, further comprising shortening a uniform resource locator for the link prior to inserting the link into the SMS text message.

14. A prescription prepayment system comprising:
a pharmacy management system at a particular store location that manages the prescription fulfillment process and sends an electronic prescription ready notification to a customer, the notification including a link;
a point of sale system at the particular store location; and
a server that manages prescription services to a customer, including managing customer prepayment for the prescription services without requiring the customer to login through a customer account, the server including a memory that stores instructions and a processor that executes the instructions to perform operations comprising:
receiving an indication that the customer has selected the link, and opening a user interface to a prepayment initiation process that is accessed by the customer via the selected link;
verifying an identity of the customer;
presenting an indication of customer prescriptions available for pickup at a particular store location;
receiving the customer's selection of one or more prescriptions from the customer prescriptions available for pickup at the particular store location;
capturing customer regulatory compliance information including the customer's e-signature for the selected one or more prescriptions;
capturing, customer payment information for payment authorization of a prescription order including the one or more prescriptions;
upon receipt of payment authorization, providing at least one of a QR code or barcode including customer payment authorization information to the customer; and
providing an order identification for the prescription order and the customer payment authorization information to the point of sale system, whereby the point of sale system is notified of the customer payment authorization information upon pickup of the prescription order having the order identification.

15. A system as in claim 14, further comprising a database that stores the order identification for the prescription order and the customer payment authorization information and maintains the order identification for the prescription order and the customer payment authorization information on a store by store basis.

16. A system as in claim 15, wherein the database periodically synchronizes the prescription order and customer payment authorization information for the particular store location with the point of sale system of the particular store location.

17. A system as in claim 14, wherein the processor executes the instructions to perform further operations comprising indicating at least one of an insurance copayment amount or a full cash price for the customer prescriptions available for pickup at the particular store location.

18. A system as in claim 14, wherein the processor executes the instructions to perform further operations comprising assigning a GUID to the customer upon verification of the identity of the customer.

19. A system as in claim 14, wherein the processor executes the instructions to perform further operations comprising receiving the customer's selection of prescriptions to remove from the customer prescriptions available for pickup at the particular store location.

20. A system as in claim 14, wherein the processor executes the instructions to perform further operations comprising receiving an indication from the customer of a person who will pick up the prescription order from the particular store location and the relationship of the person to the customer.

21. A system as in claim 14, wherein the processor executes the instructions to perform further operations comprising receiving documentation of pharmaceutical care responses from the customer and tying the documentation of pharmaceutical care responses to the order identification for the prescription order.

22. A system as in claim 14, wherein the point of sale system comprises a display, the point of sale system receiving a scan of the at least one QR code or barcode and flagging from the scan for presentation on the display of the point of sale system that the customer has prepaid for the prescription order having the order identification.

23. A system as in claim 22, wherein the point of sale system presents a PAY button to the display when the scan of the at least one QR code or barcode indicates that the customer has prepaid for the prescription order having the order identification.

24. A system as in claim 14, wherein the point of sale system provides access to a pre-paid order management portal application that displays active prescription orders for the particular store location.

25. A system as in claim 14, further comprising a messaging server that sends the electronic prescription ready notification to the customer as an SMS text message or an email including the link.

26. A system as in claim 25, further comprising a uniform resource locator (URL) shortener that shortens a URL for the link prior to inserting the link into the SMS text message.
